(19) United States
(12) Patent Application Publication
(10) Pub. No.: US 2026/0174717 A1
(43) Pub. Date: Jun. 25, 2026

(54) ULOTARONT FOR TREATING ANXIETY AND ASSOCIATED CONDITIONS

(71) Applicant: Sumitomo Pharma America, Inc., Marlborough, MA (US)

(72) Inventors: Nina DEDIC, Marlborough, MA (US); Robert HAYES, Marlborough, MA (US); Justine KENT, Marlborough, MA (US)

(73) Assignee: Sumitomo Pharma America, Inc., Marlborough, MA (US)

(21) Appl. No.: 19/124,358

(22) PCT Filed: Oct. 26, 2023
(86) PCT No.: PCT/US2023/077818

§ 371 Date: Apr. 25, 2025

Related U.S. Application Data
(60) Provisional application No. 63/381,327, filed on Oct. 28, 2022.

(51) Int. Cl. A61K 31/198; A61K 9/00; A61K 38/00; A61K 45/06

(52) U.S. Cl. CPC A61K 31/198; A61K 9/0053; A61K 38/00; A61K 45/06

(57) ABSTRACT

Neuropsychiatric treatments, including methods, regimens, and interventions to treat anxiety and associated symptoms, based on ulotaront administration.

### CROSS-REFERENCE TO RELATED APPLICATIONS

The present application claims priority to U.S. provisional application 63/381,327, filed Oct. 28, 2022, the contents of which are incorporated herein by reference in their entirety.

### FIELD

The present disclosure relates to pharmacological neuropsychiatric treatments, and to methods, regimens, and interventions to treat anxiety and associated conditions based on ulotaront administration.

### BACKGROUND

Generalized anxiety disorder (GAD), a prevalent and often recurrent disorder, is associated with significant medical and psychiatric morbidity and mortality, functional disability, and health care costs. A recent survey indicates GAD has a 12-month prevalence of 2.9% and a lifetime prevalence of 6.2% in the US adult population (Kessler 2012). In a global survey GAD had a 12-month prevalence of 1.8% and lifetime prevalence of 3.7% with both prevalence and functional impact greater in high-income countries compared to low-income countries (Ruscio 2017). Generally, anxiety disorders are a significant contributor to decreased work performance and increased health care resource utilization accounting for substantial economic impact (Wittchen 2002; DuPont 1996; Greenberg 1999). GAD in particular results in substantial impact and economic burden to individuals and society (Pollack 2009).

Pharmacotherapy for GAD typically consists of antidepressants (e.g., selective serotonin reuptake inhibitors (SSRI) and serotonin and norepinephrine reuptake inhibitors (SNRI)) with benzodiazepines and other agents used as an augmentation when required (Garakani 2020). SSRIs and SNRIs generally have modest success controlling GAD symptoms, but they carry substantial side effects which are an important limiter of pharmacotherapy compliance and, ultimately, treatment outcomes (Kong 2020; Garakani 2020). While benzodiazepines have shown immediate and robust efficacy against GAD symptoms (Gomez 2018; Garakani 2020), their abuse and dependence liability limits their use to relatively short intervals in most treatment guidelines (Balon 2020). These factors, coupled with the fact that GAD is a chronic disorder, lead to a substantial numbers of patients who either do not respond to or do not tolerate first line treatment (Bandelow 2008 Goodwin 2002; Altamura 2008; Allgulander 2002; Baldwin 2005). Relatedly, remission rates are low in GAD (Yonkers 1996) and the probability of relapse after remission is 27-39% by 3 years (Yonkers 2000).

For these reasons, there exists a substantial unmet need for new agents for the treatment of GAD that alleviate symptoms and improve response and remission rates but are not burdened with the side effect profile associated with available neuropsychiatric medications and lack the abuse and dependence liability of benzodiazepines and other neuropsychiatric medications.

### SUMMARY

In one embodiment, the disclosure provides a method of treating an anxiety disorder in a non-schizophrenic human subject in need thereof who is no more than mildly depressed, comprising administering to the subject a therapeutically effective amount of ulotaront or a pharmaceutically acceptable salt thereof, wherein the treatment results in a reduction in the subject's HAM-A total score compared to placebo.

In another embodiment, the disclosure provides a method of treating an anxiety disorder in a human subject in need thereof having a HAM-A total score≥20, comprising administering to the subject a therapeutically effective amount of ulotaront or a pharmaceutically acceptable salt thereof, wherein the treatment results in a reduction in the subject's HAM-A total score compared to placebo.

Another embodiment provides a method of treating an anxiety disorder in a human subject in need thereof having a HAM-A total score≥20, a HAM-A anxious mood score≥2, and a HAM-A tension score of ≥2, comprising administering to the subject a therapeutically effective amount of ulotaront or a pharmaceutically acceptable salt thereof, wherein the treatment results in a reduction in the subject's HAM-A total score compared to placebo.

Another embodiment provides a method of treating tension in a human subject also suffering from a neuropsychiatric disorder, comprising administering to the subject a therapeutically effective amount of ulotaront or a pharmaceutically acceptable salt thereof, wherein the treatment results in a reduction in the subject's HAM-A total score compared to placebo.

Additional advantages of the disclosure are set forth in part in the description which follows, and in part will be obvious from the description, or may be learned by practice of the disclosure. The advantages of the disclosure will be realized and attained by means of the elements and combinations particularly pointed out in the appended claims. It is to be understood that both the foregoing general description and the following detailed description are exemplary and explanatory only and are not restrictive of the disclosure, as claimed.

### DETAILED DESCRIPTION

All published documents cited herein are hereby incorporated herein by reference in their entirety.

### Use of Terms

As used herein, the singular forms “a”, “an” and “the” are intended to include the plural forms as well, unless the context clearly indicates otherwise.

Unless otherwise specified, the word “includes” (or any variation thereon, e.g., “include”, “including”, etc.) is intended to be open-ended. For example, “A includes 1, 2 and 3” means that A includes, but is not limited to, 1, 2 and 3.

As used in this specification and in the claims which follow, the word “comprise” and variations of the word, such as “comprising” and “comprises,” means “including but not limited to,” and is not intended to exclude, for example, other additives, components, integers or steps. When an element is described as comprising a plurality components, steps or conditions, it will be understood that the element can also be described as comprising any combination of such plurality, or “consisting of” or “consisting essentially of” the plurality or combination of components, steps or conditions.

When ranges are given by specifying the lower end of a range separately from the upper end of the range, or particular numerical values are specified, it will be understood that a range can be defined by selectively combining any of the lower end variables, upper end variables, and particular numerical values that is mathematically possible. When ranges are stated as extending from one endpoint to another endpoint, it will be understood that the two endpoints are included in the range. However, it will also be understood that a from/to range also includes an embodiment in which the range is defined as between the two specified endpoints, and that the term “between” can be substituted for the “from/to” language to omit the endpoints from the range.

The present disclosure describes various embodiments. A person of ordinary skill in the art reviewing the disclosure will readily recognize that various embodiments can be combined in any variation. For example, embodiments of the disclosure include treatment of various disorders, patient populations, administrations of dosage forms, at various dosages, minimization of various adverse events, and improvements in various efficacy measures, etc. Any combinations of various embodiments are within the scope of the disclosure.

When published test methodologies and diagnostic instruments are referred to herein, it will be understood that the test methodology or diagnostic instrument is performed based on the version in effect on Oct. 1, 2022, unless otherwise stated to the contrary herein. This is true even when the methodology or instrument is defined herein based on a publication reporting an earlier version.

Unless defined otherwise, all technical and scientific terms used herein have the same meaning as commonly understood by one of ordinary skill in the art to which this disclosure belongs.

### Definitions

As used herein, “administering” or “administration” of ulotaront, or a pharmaceutically acceptable salt thereof, encompasses the delivery to a subject of ulotaront, or a pharmaceutically acceptable salt thereof, or a prodrug or other pharmaceutically acceptable derivative thereof, using any suitable formulation or route of administration, e.g., as described herein.

The term “atypical antidepressants” refers to antidepressants that do not fit into the usual classifications for antidepressants (e.g., SSRI, SSNI, tricyclics and MOAIs). Examples include bupropion (Wellbutrin®), vilazodone (Viibryd®), vortioxetine (Trintellix®), nefazodone, NASSAs (noradrenaline and specific serotonergic antidepressants) such as mirtazapine (Remeron®), SARIs (serotonin antagonists and reuptake inhibitors such as trazodone (Molipaxin®), and NARIs (noradrenaline reuptake inhibitor) such as reboxetine.

The “CGI-C” (Clinical Global Impression-Change) Scale measures efficacy of drug treatment by rating the subject's total change from the initiation of drug therapy whether or not it is due entirely to drug treatment. Response choices include: 1=very much improved, 2=much improved, 3=minimally improved, 4=no change, 5=minimally worse, 6=much worse, and 7=very much worse.

The “CGI-S” (Clinical Global Impression-Severity) Scale is a standardized, clinician-administered global rating scale that measures disease severity on a 7-point Likert scale. A higher score on the CGI-S represents a higher severity of disease. To perform this assessment, the rater or investigator answers the following question: “Considering your total clinical experience with this particular population, how mentally ill is the patient at this time?” Response choices include: 1=normal, not at all ill; 2=borderline mentally ill; 3=mildly ill; 4=moderately ill; 5=markedly ill; 6=severely ill; and 7=among the most extremely ill patients.

As used herein, a “clinically significant” or “clinically meaningful” improvement can mean an improvement which is both statistically significant and meaningful from a patient's, clinician's, or caregiver's perspective, typically based on a static measure such as CGI-S or a retrospective evaluation of improvement such as the CGI-C, as described generally in various publications of the United States Food and Drug Administration, including FDA 2018, FDA 2019, and FDA 2020. When a treatment or benefit is described herein, it will be understood that the treatment or benefit preferably shows clinically significant efficacy in a population of patients to a degree of statistical significance.

“Cyclic antidepressants” include tricyclics and tetracyclics, and typically function by blocking the reabsorption (reuptake) of the neurotransmitters serotonin and norepinephrine, increasing the levels of these two neurotransmitters in the brain. Examples include amitriptyline, amoxapine, desipramine (Norpramin®), doxepin, imipramine (Tofranil®), nortriptyline (Pamelor®), protriptyline, trimipramine, and maprotiline.

As used herein, “delaying” development of a disorder means to defer, hinder, slow, stabilize, and/or postpone development of the disorder. Delay can be of varying lengths of time, depending on the history of the disease and/or the individual being treated.

“DSM-5” refers to the Diagnostic and Statistical Manual of Mental Disorders, Fifth Edition. Terms used herein can be defined by reference to DSM-5 when necessary to give life and meaning to the term. When a person is defined in this document based on DSM-5, it will be understood that the person need not have been diagnosed using the criteria in DSM-5, but that the person would meet the criteria specified in DSM-5 if so diagnosed.

The Hamilton Anxiety Rating Scale (HAM-A) is a clinician-administered rating scale developed to quantify the severity of anxiety symptomatology. It consists of 14 items, each defined by a series of symptoms. Each item is rated on a 5-point (0-4) scale, with higher scores indicating greater severity. (Hamilton 1959).

The Healthcare Resource Utilization (HCRU) is the quantification or description of the use of services by persons for the purpose of preventing and curing health problems, promoting maintenance of health and well-being, or obtaining information about one's health status and prognosis. The HCRU includes questions regarding the numbers of physician office visits, emergency room (ER) visits, hospitalizations, and length of hospital stays for any reason and for care related to GAD during the past 1 month.

“MAOIs” refers to the monoamine oxidase inhibitor class of antidepressants. Examples include isocarboxazid (Marplan®), phenelzine (Nardil®), selegiline (Emsam®), and tranylcypromine (Parnate®).

The Medication Satisfaction Questionnaire (MSQ) is a single-item, subject-rated, rater administered questionnaire that requires the subject to use a 7-point, Likert-type scale to rate how satisfied they are with their medication. The subject is asked the following question: “Overall, how satisfied are you with your current GAD medication?” Subjects select 1 of 7 potential responses based on their level of satisfaction, from (1) extremely dissatisfied to (7) extremely satisfied.

The Montgomery-Åsberg Depression Rating Scale (MADRS) is a clinician-rated assessment of the subject's level of depression. The measure contains 10 items that measure apparent and reported sadness, inner tension, reduced sleep and appetite, difficulty concentrating, lassitude, inability to feel, pessimistic thoughts, and suicidal thoughts. Each item is scored in a range of 0 to 6 points, with higher scores indicating increased depressive symptoms. (Montgomery 1979).

“Pharmaceutically acceptable” or “physiologically acceptable” refer to compounds, salts, compositions, dosage forms and other materials which are useful in preparing a pharmaceutical composition that is suitable for veterinary or human pharmaceutical use.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19. Pharmaceutically acceptable salts of ulotaront include those derived from suitable inorganic and organic acids and bases.

Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemi sulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like. Although pharmaceutically acceptable counter ions will be preferred for preparing pharmaceutical formulations, other anions (X) are quite acceptable as synthetic intermediates. Thus, X may be pharmaceutically undesirable anions, such as iodide, oxalate, trifluoromethanesulfonate and the like, when such salts are chemical intermediates.

As used herein, the term “pharmaceutically acceptable excipient” includes, without limitation, any binder, filler, adjuvant, carrier, excipient, glidant, sweetening agent, diluent, preservative, dye/colorant, flavor enhancer, surfactant, wetting agent, dispersing agent, suspending agent, stabilizer, isotonic agent, solvent, emulsifier, anti-caking agent, flavor, desiccants, plasticizers, disintegrants, lubricant, polymer matrix system, and polishing agents, which has been approved by or is otherwise acceptable to the United States Food and Drug Administration upon proper qualification for use in humans or domestic animals.

As used herein, “prevention” or “preventing” refers to a regimen that protects against the onset of the disorder such that the clinical symptoms of the disorder do not develop. Accordingly, “prevention” relates to administration of a therapy to a subject before signs of the diseases are detectable in the subject (for example, administration of a therapy in the absence of a detectable symptom of the disorder). The subject may be an individual at risk of developing the disorder.

The Sheehan Disability Scale (SDS) SDS is a clinician-assisted scale/composite of three items designed to measure the extent to which three major sectors in the subject's life are impaired by psychiatric or medical symptoms. This anchored visual analog scale uses spatiovisual, numeric, and verbal descriptive anchors simultaneously to assess disability across three domains: work, social life, and family life. The subject rates the extent to which his or her 1) work, 2) social life or leisure activities, and 3) home life or family responsibilities are impaired by his or her symptoms on an 11-point visual analogue scale ranging from 0 to 10. There are verbal descriptors for the points on the scale as well as numerical scores that provide more precise levels of the verbal descriptors. The three items may be summed into a single dimensional measure of global functional impairment that ranges from 0 (unimpaired) to 30 (highly impaired).

The term “selecting” refers to the act of choosing from a number or group by fitness or preference. In the context of the current disclosure, ulotaront is selected from a group of generally recognized neuropsychiatric medications, for the treatment of any of the neuropsychiatric conditions described herein.

The 36-Item Short Form Questionnaire (“SF-36”) is a self-reported questionnaire with a standard recall period of 4 weeks which measures generic health-related quality of life on 2 broad domains, physical and mental composites, across 8 health domain scales: physical functioning, pain, role physical, general health, vitality/fatigue, social functioning, role emotional, and mental health. The SF-36 uses norm-based scoring to generate scores on a scale of 1 to 100 where lower scores on the physical component summary and mental component summary represent a lower health-related quality of life, and a score of 50 references the normative data derived from surveys of representative samples of US general population.

As used herein, the term “significantly” refers to a level of statistical significance. The level of statistical significance can be p<0.1, p<0.05, p<0.01, p<0.005, or p<0.001. Unless otherwise specified, the level of statistical significance when the term “significant,” “significantly,” or other variations of the term are used is p<0.05. When a measurable result or effect is expressed or identified herein, it will be understood that the result or effect is preferably evaluated based upon its statistical significance relative to a baseline such as placebo. In like manner, when a treatment or benefit is described herein, it will be understood that the treatment or benefit preferably shows efficacy in a population of patients to a degree of statistical significance.

“SNRIs” (Serotonin-norepinephrine Reuptake Inhibitors) include, without limitation, desvenlafaxine (Pristiq®), duloxetine (Cymbalta®), levomilnacipran (Fetzima®), and venlafaxine (Effexor® XR), and their pharmaceutically acceptable salts.

“SSRIs” (Selective Serotonin Reuptake Inhibitors) include, without limitation, citalopram (Celexa®), escitalopram (Lexapro®), fluoxetine (Prozac®), paroxetine (Paxil®, Pexeva®), and sertraline (Zoloft®), and their pharmaceutically acceptable salts.

The Structured Clinical Interview for DSM-5 Axis I Disorders—Clinical Trials Version (SCID-5-CT) is a modified version of the SCID developed for use in clinical trials. It is a semi-structured interview for the purpose of making a DSM-5 diagnosis (First 2015).

As used herein, “subject” or “patient” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or other primates (e.g., cynomolgus monkeys, rhesus monkeys); mammals, including commercially relevant mammals such as cattle, pigs, horses, sheep, goats, cats, and/or dogs; and/or birds, including commercially relevant birds such as chickens, ducks, geese, quail, and/or turkeys.

As used herein, the term “therapeutically effective amount” or “effective amount” refers to an amount that is effective to elicit the desired biological or medical response, including the amount of a compound that, when administered to a subject for treating a disorder, is sufficient to accomplish such treatment of the disorder. The effective amount will vary depending on the disorder, and its severity, and the age, weight, etc. of the subject to be treated. The effective amount may be in one or more doses (for example, a single dose or multiple doses may be required to achieve the desired treatment endpoint). An effective amount may be considered to be given in an effective amount if, in conjunction with one or more other agents, a desirable or beneficial result may be or is achieved. Suitable doses of any co-administered compounds may optionally be lowered due to the combined action, additive or synergistic, of the compound.

As used herein, the terms “treatment,” “treat,” and “treating” refer to reversing, alleviating, delaying the onset of, or inhibiting the progress of a disease or disorder, or one or more symptoms thereof, including but not limited to therapeutic benefit. In some embodiments, treatment is administered after one or more symptoms have developed, for example, acute exacerbation of symptoms. In some embodiments, treatment may be administered in the absence of symptoms. For example, treatment may be administered to a subject prior to the onset of symptoms (e.g., in light of a history of symptoms and/or in light of genetic or other susceptibility factors). Treatment may also be continued after symptoms have resolved, for example to prevent or delay their recurrence.

Therapeutic benefit includes eradication and/or amelioration of the underlying disorder being treated; it also includes the eradication and/or amelioration of one or more of the symptoms associated with the underlying disorder such that an improvement is observed in the subject, notwithstanding that the subject may still be afflicted with the underlying disorder.

In some embodiments, “treatment” or “treating” includes one or more of the following: (a) inhibiting the disorder (for example, decreasing one or more symptoms resulting from the disorder, and/or diminishing the extent of the disorder); (b) slowing or arresting the development of one or more symptoms associated with the disorder (for example, stabilizing the disorder and/or delaying the worsening or progression of the disorder); and/or (c) relieving the disorder (for example, causing the regression of clinical symptoms, ameliorating the disorder, delaying the progression of the disorder, and/or increasing quality of life.)

“Ulotaront” (a/k/a SEP-363856, SEP-856), as referred to herein for use in the methods of the present disclosure, has the chemical name(S)-(4,5-dihydro-7H-thieno[2,3-c]pyran-7-yl)-N-methylmethanamine (which may be abbreviated as “(S)-TPMA”). Ulotaront has the following structure:

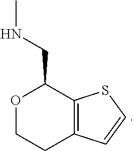

Unless stated otherwise, or unless context requires otherwise, for purposes of this disclosure, the term “ulotaront,” standing alone, includes the free form of ulotaront and also includes its pharmaceutically acceptable salts, hydrates, solvates, amorphous and crystalline forms. When the free form is intended, or any other form or salt is specifically intended, it will be stated as such expressly.

Ulotaront can be used in the methods described herein as the free base or in the form of a pharmaceutically acceptable salt. In preferred embodiments, a hydrochloric acid (HCl) salt of ulotaront is used in the methods described herein. Ulotaront, or a pharmaceutically acceptable salt thereof, including its HCl crystalline forms, can be obtained according to the production methods described in PCT Patent Publication No. WO2011/069063 (U.S. Pat. No. 8,710,245, issued Apr. 29, 2014) or PCT Patent Publication No. WO2019/161238, which are incorporated herein by reference in entirety and for all purposes, or a method analogous thereto.

Also provided herein are pharmaceutical compositions and dosage forms, comprising ulotaront, or a pharmaceutically acceptable salt thereof, and one or more pharmaceutically acceptable excipients. Compositions and dosage forms provided herein may further comprise one or more additional active ingredients. Ulotaront, or a pharmaceutically acceptable salt thereof, may be administered as part of a pharmaceutical composition as described herein.

### Discussion

In one embodiment, the disclosure provides a method of treating an anxiety disorder in a non-schizophrenic human subject in need thereof who is no more than mildly depressed, comprising administering to the subject a therapeutically effective amount of ulotaront or a pharmaceutically acceptable salt thereof, wherein the treatment results in a reduction in the subject's HAM-A total score compared to placebo.

In another embodiment, the disclosure provides a method of treating an anxiety disorder in a human subject in need thereof having a HAM-A total score≥20, comprising administering to the subject a therapeutically effective amount of ulotaront or a pharmaceutically acceptable salt thereof, wherein the treatment results in a reduction in the subject's HAM-A total score compared to placebo.

Another embodiment provides a method of treating an anxiety disorder in a human subject in need thereof having a HAM-A total score≥20, a HAM-A anxious mood score≥2, and a HAM-A tension score of ≥2, comprising administering to the subject a therapeutically effective amount of ulotaront or a pharmaceutically acceptable salt thereof, wherein the treatment results in a reduction in the subject's HAM-A total score compared to placebo.

Another embodiment provides a method of treating tension in a human subject also suffering from a neuropsychiatric disorder comprising administering to the subject a therapeutically effective amount of ulotaront or a pharmaceutically acceptable salt thereof, wherein the treatment results in a reduction in the subject's HAM-A total score compared to placebo.

In various embodiments, the treatment accomplishes various degrees of results. Thus, in some embodiments, the treatment results in a clinically significant reduction in the subject's HAM-A total score compared to placebo. In some embodiments, the treatment also results in a clinically significant reduction in the subject's CGI-S score compared to placebo. In some embodiments, the treatment results in clinically significant reductions in the subject's HAM-A total score and CGI-S score compared to placebo. In further embodiments, the treatment results in clinically significant reductions in the subject's HAM-A total score and CGI-S score compared to placebo eight weeks after commencing the administration. In still further embodiments, the treatment results in clinically significant reductions in the subject's HAM-A total score and CGI-S score compared to placebo four and eight weeks after commencing the administration.

Other embodiments are defined based on the numerical benefits achieved by the method. Thus, in some embodiments, the treatment reduces the subject's HAM-A total score to ≤12, ≤11, ≤10, ≤9, ≤8, ≤7, or ≤6. In some embodiments, the treatment reduces the subject's HAM-A total score by ≥40%, ≥50%, or ≥60%. In some embodiments, the treatment reduces the subject's CGI-S score by ≥1 or ≥2 or ≥3.

When a treatment is said to produce a clinically significant treatment effect, it will be understood that the treatment can also be said, in other embodiments, to produce a reduction in the subject's CGI-S score by ≥2. When a treatment is said to produce a clinically significant treatment effect, it will be understood that the treatment can also be said, in other embodiments, to produce a reduction in the subject's CGI-S score by ≥50%.

Given ulotaront's unique behavioral signatures (as reported by Dedic 2019), the human clinical results reported in PCT Patent Publication No. WO 2020/118032, and ulotaront's novel mechanism of action and receptor binding profile, ulotaront can be expected to treat neuropsychiatric disorders with a high degree of safety and efficacy. Thus, in one embodiment, ulotaront is administered to patients also suffering from a neuropsychiatric disorder selected from psychoses, depression, pain, cognition, mood disorders, and anxiety.

In another embodiment, the neuropsychiatric disorders include schizophrenia, schizophrenia spectrum disorder, acute schizophrenia, chronic schizophrenia, NOS schizophrenia, schizoid personality disorder, schizotypal personality disorder, delusional disorder, psychosis, psychotic disorder, brief psychotic disorder, shared psychotic disorder, psychotic disorder due to a general medical condition, drug-induced psychosis (e.g., cocaine, alcohol, amphetamine), psychoaffective disorder, excitative psychosis, organic or NOS psychosis, dyskinesias, mood disorder, anxiety, affective disorders (e.g., depression, e.g., major depressive disorder and dysthymia; bipolar disorder, e.g., bipolar depressive disorder, manic disorder, seasonal affective disorder), pain (e.g., neuropathic pain, sensitization accompanying neuropathic pain, and inflammatory pain), cognitive impairment, and movement disorder.

In one embodiment, the neuropsychiatric disorder is selected from schizophrenia, depression, and anxiety. In another embodiment, the neuropsychiatric disorder is schizophrenia. In another embodiment, the neuropsychiatric disorder is bipolar depression (particularly MDD as defined by DSM-5 and, more particularly, AMDD). In still another embodiment the neuropsychiatric disorder is anxiety (for example, generalized anxiety disorder (GAD) as defined by DSM-5). In one embodiment, the neuropsychiatric disorder is an anxiety disorder.

In some embodiments, the treating comprises remitting the tension or anxiety disorder. In some embodiments, the treating comprises a complete response to the tension or anxiety disorder. In other embodiments, the treating comprises both remitting the tension or anxiety disorder and a complete response to the tension or anxiety disorder.

In one embodiment, the subject is treatment naïve.

In one embodiment, the subject has failed to adequately respond to prior buspirone or antidepressant therapy.

In one embodiment, the subject has failed to adequately tolerate prior buspirone or antidepressant therapy.

As exemplary antidepressant therapies, SSRIs, SNRIs, cyclic antidepressants, atypical antidepressants, and MAOIs can be mentioned, although any molecule exhibiting an antidepressant mechanism of action is intended.

Anxiety disorders treated by the methods of this disclosure include, without limitation, generalized anxiety disorder (GAD), social anxiety disorder (SAD), obsessive-compulsive disorder (OCD), post-traumatic stress disorder (PTSD) and panic disorder. In one embodiment, the anxiety disorder is generalized anxiety disorder (GAD).

The methods of the disclosure, when used to treat anxiety disorder, also can be used to treat tension. Thus, when the patient also suffers from tension, the methods can further comprise treating the tension. In one embodiment, the patient suffers from tension, and the method comprises treating the tension to a clinically significant degree.

In like manner, the methods of the disclosure, when used to treat tension, also can be used to treat anxiety disorder. Thus, when the patient also suffers from anxiety disorder, the methods can further comprise treating the anxiety disorder.

In one embodiment, the methods of the disclosure are used to treat non-schizophrenic subjects. In other embodiments, the methods are used to treat subjects who are no more than mildly depressed.

When a score is provided (including, but not limited to, HAM-A, MADRS, CGI-S, etc.), unless otherwise indicated, the score is that determined at the initiation of therapy. For instance, “A method of treating an anxiety disorder in a human subject in need thereof having a HAM-A total score≥20 . . . ” indicates that the subject's HAM-A total score is ≥20 at the initiation of therapy.

The methods of the disclosure can also be practiced based on a subject's score on HAM-A. In some embodiments, the subject has a HAM-A total score≥20. In some embodiments, the subject has a HAM-A total score≥20, ≥22.5, ≥25, ≥27.5, ≥30, ≥32.5, or ≥35.

In some embodiments, the subject has a HAM-A anxious mood score≥2. In some embodiments, the subject has a HAM-A anxious mood score≥2, ≥4, or =4.

In some embodiments, the subject has a HAM-A tension score of ≥2. In some embodiments, the subject has a HAM-A tension score≥2, ≥3, or =4.

In other embodiments, the subject has a HAM-A total score≥20, a HAM-A anxious mood score≥2, and a HAM-A tension score of ≥2.

The methods of the disclosure also can be practiced based on a subject's MADRS score. In some embodiments, the subject has a MADRS total score≤22. In some embodiments, the subject has a MADRS total score≤22, ≤20, ≤18, ≤16, ≤14, ≤12, or ≤10. When the term “mildly depressed” is used herein, it will be understood that a MADRS score ranging from 7 to 19 generally indicates “mild depression” when using the MADRS scoring system. However, for purposes of this disclosure, the term mildly depressed includes patients having a MADRS score≤22. A subject who is no more than “mildly depressed” can thus be alternatively defined as having a MADRS total score≤22, ≤19, ≤16, ≤14, ≤12, ≤10, or ≤8. Alternatively, the subject can be described based on a non-depressed MADRS score (i.e., <7), or a mildly depressed MADRS score (i.e., from 7 to 22 or from 7 to 19).

In some embodiments, the subject has a MADRS inner tension score≥2, ≥3, ≥4, ≥5, or =6.

The methods of the disclosure also can be practiced based on the subject's CGI-S score. In some embodiments, the subject has a CGI-S≥4. In some embodiments, the subject has a CGI-S score≥4, and the treatment reduces the subject's CGI-S score by ≥2 points. In some embodiments, the subject has a CGI-S score≥4, and the treatment reduces the CGI-S score to ≤3.

The anxiety disorders of the disclosure can be characterized based on one or a combination of feelings or symptoms. In one embodiment, the anxiety disorder comprises one or a combination of feelings or symptoms selected from anxious mood, tension, fears, insomnia, intellectual, depressed mood, somatic (muscular), somatic (sensory), cardiovascular symptoms, respiratory symptoms, gastrointestinal symptoms, genitourinary symptoms, autonomic symptoms, and anxious behavior.

In one embodiment, the anxiety disorder comprises one or more anxious mood feelings or symptoms selected from worries, anticipation of the worst, fearful anticipation, and irritability. In another embodiment, the subject has a HAM-A total score≥20, ≥22.5, ≥25, ≥27.5, ≥30, ≥32.5, or ≥35, and a HAM-A anxious mood score of ≥2, ≥3, or =4.

In one embodiment, the anxiety disorder comprises one or more tension feelings or symptoms selected from feelings of tension, fatigability, startle response, moved to tears easily, trembling, feelings of restlessness, and inability to relax. In another embodiment, the subject has a HAM-A total score≥20, ≥22.5, ≥25, ≥27.5, ≥30, ≥32.5, or ≥35, and a HAM-A tension score of ≥2, ≥3, or =4.

In one embodiment, the anxiety disorder comprises one or more fears selected from fear of dark, fear of strangers, fear of being left alone, fear of animals, fear of traffic, and fear of crowds. In another embodiment, the subject has a HAM-A total score≥20, ≥22.5, ≥25, ≥27.5, ≥30, ≥32.5, or ≥35, and a HAM-A fears score of ≥2, ≥3, or =4.

In one embodiment, the anxiety disorder comprises one or more insomnia feelings or symptoms selected from difficulty in falling asleep, broken sleep, unsatisfying sleep and fatigue on waking, dreams, nightmares, and night terrors. In another embodiment, the subject has a HAM-A total score≥20, ≥22.5, ≥25, ≥27.5, ≥30, ≥32.5, or ≥35, and a HAM-A insomnia score of ≥2, ≥3, or =4.

In one embodiment, the anxiety disorder comprises one or more intellectual feelings or symptoms selected from difficulty in concentration and poor memory. In another embodiment, the subject has a HAM-A total score≥20, ≥22.5, ≥25, ≥27.5, ≥30, ≥32.5, or ≥35, and a HAM-A intellectual score of ≥2, ≥3, or =4.

In one embodiment, the anxiety disorder comprises one or more depressed mood feelings or symptoms selected from loss of interest, lack of pleasure in hobbies, depression, early waking, and diurnal swing. In another embodiment, the subject has a HAM-A total score≥20, ≥22.5, ≥25, ≥27.5, ≥30, ≥32.5, or ≥35, and a HAM-A depressed mood score of ≥2, ≥3, or =4.

In one embodiment, the anxiety disorder comprises one or more somatic (muscular) feelings or symptoms selected from pains and aches, twitching, stiffness, myoclonic jerks, grinding of teeth, unsteady voice, and increased muscular tone. In another embodiment, the subject has a HAM-A total score≥20, ≥22.5, ≥25, ≥27.5, ≥30, ≥32.5, or ≥35, and a HAM-A somatic (muscular) score of ≥2, ≥3, or =4.

In one embodiment, the anxiety disorder comprises one or more somatic (sensory) feelings or symptoms selected from tinnitus, blurring of vision, hot and cold flushes, feelings of weakness, and pricking sensation. In another embodiment, the subject has a HAM-A total score≥20, ≥22.5, ≥25, ≥27.5, ≥30, ≥32.5, or ≥35, and a HAM-A somatic (sensory) score of ≥2, ≥3, or =4.

In one embodiment, the anxiety disorder comprises one or more cardiovascular symptoms selected from tachycardia, palpitations, pain in chest, throbbing of vessels, fainting feelings, and missing beat. In another embodiment, the subject has a HAM-A total score≥20, ≥22.5, ≥25, ≥27.5, ≥30, ≥32.5, or ≥35, and a HAM-A cardiovascular score of ≥2, ≥3, or =4.

In one embodiment, the anxiety disorder comprises one or more respiratory symptoms selected from pressure or constriction in chest, choking feelings, sighing, and dyspnea. In another embodiment, the subject has a HAM-A total score≥20, ≥22.5, ≥25, ≥27.5, ≥30, ≥32.5, or ≥35, and a HAM-A respiratory score of ≥2, ≥3, or =4.

In one embodiment, the anxiety disorder comprises one or more gastrointestinal symptoms selected from difficulty in swallowing, wind abdominal pain, burning sensations, abdominal fullness, nausea, vomiting, borborygmi, looseness of bowels, loss of weight, and constipation. In another embodiment, the subject has a HAM-A total score≥20, ≥22.5, ≥25, ≥27.5, ≥30, ≥32.5, or ≥35, and a HAM-A gastrointestinal score of ≥2, ≥3, or =4.

In one embodiment, the anxiety disorder comprises one or more genitourinary symptoms selected from frequency of micturition, urgency of micturition, amenorrhea, menorrhagia, development of frigidity, premature ejaculation, loss of libido, and impotence. In another embodiment, the subject has a HAM-A total score≥20, ≥22.5, ≥25, ≥27.5, ≥30, ≥32.5, or ≥35, and a HAM-A genitourinary score of ≥2, ≥3, or =4.

In one embodiment, the anxiety disorder comprises one or more autonomic symptoms selected from dry mouth, flushing, pallor, tendency to sweat, giddiness, tension headache, and raising of hair. In another embodiment, the subject has a HAM-A total score≥20, ≥22.5, ≥25, ≥27.5, ≥30, ≥32.5, or ≥35, and a HAM-A autonomic score of ≥2, ≥3, or =4.

In one embodiment, the anxiety disorder comprises one or more anxious behaviors selected from fidgeting, restlessness or pacing, tremor of hands, furrowed brow, strained face, sighing or rapid respiration, facial pallor, or swallowing. In another embodiment, the subject has a HAM-A total score≥20, ≥22.5, ≥25, ≥27.5, ≥30, ≥32.5, or ≥35, and a HAM-A behavior at interview score of ≥2, ≥3, or =4.

The therapeutically effective amount of ulotaront can be described variously as 10-150 mg/day or 25-150 mg/day or 25-100 mg/day or 50-125 mg/day or 50-100 mg/day or 50-75 mg/day, administered orally. Alternatively, the therapeutically effective amount can be described as 10 mg/day, 15 mg/day, 20 mg/day, 25 mg/day, 50 mg/day, 75 mg/day, 100 mg/day, 125 mg/day, or 150 mg/day, orally administered. In any of the embodiments of the disclosure, the therapeutically effective amount can be administered once daily in the fed or fasted state. The ulotaront can also be administered as the hydrochloride salt.

Preferred aspects of the disclosure can be defined based on Embodiments AA through CM below:

[Embodiment AA] A method of treating an anxiety disorder in a non-schizophrenic human subject in need thereof who is no more than mildly depressed comprising administering to the subject a therapeutically effective amount of ulotaront or a pharmaceutically acceptable salt thereof, wherein the treatment results in a reduction in the subject's HAM-A total score compared to placebo.

[Embodiment AB] A method of treating an anxiety disorder in a human subject in need thereof having a HAM-A total score≥20 comprising administering to the subject a therapeutically effective amount of ulotaront or a pharmaceutically acceptable salt thereof, wherein the treatment results in a reduction in the subject's HAM-A total score compared to placebo.

[Embodiment AC] A method of treating an anxiety disorder in a human subject in need thereof having a HAM-A total score≥20, a HAM-A anxious mood score≥2, and a HAM-A tension score of ≥2, comprising administering to the subject a therapeutically effective amount of ulotaront or a pharmaceutically acceptable salt thereof, wherein the treatment results in a reduction in the subject's HAM-A total score compared to placebo.

[Embodiment AD] The method of any of Embodiments AA-AC, wherein the treatment results in a clinically significant reduction in the subject's HAM-A total score compared to placebo.

[Embodiment AE] The method of any of Embodiments AA-AC, wherein the treatment also results in a clinically significant reduction in the subject's CGI-S score compared to placebo.

[Embodiment AF] The method of any of Embodiments AA-AC, wherein the treatment results in clinically significant reductions in the subject's HAM-A total score and CGI-S score compared to placebo eight weeks after commencing the administration.

[Embodiment AG] The method of any of Embodiments AA-AC, wherein the treatment results in clinically significant reductions in the subject's HAM-A total score and CGI-S score compared to placebo four and eight weeks after commencing the administration.

[Embodiment AH] The method of any of Embodiments AA-AG, wherein the treatment reduces the subject's HAM-A total score to ≤7.

[Embodiment AI] The method of any of Embodiments AA-AH, wherein the treatment reduces the subject's HAM-A total score by ≥50%.

[Embodiment AJ] The method of any of Embodiments AA-AI, wherein the treatment reduces the subject's CGI-S score by ≥1 or ≥2.

[Embodiment AK] The method of any of Embodiments AA-AI, wherein the treatment reduces the subject's CGI-S score to ≤3.

[Embodiment AL] The method of any of Embodiments AA-AI, wherein the treatment reduces the subject's CGI-S score to ≤2.

[Embodiment AM] The method of any of Embodiments AB-AL, wherein the subject is non-schizophrenic.

[Embodiment AN] The method of any of Embodiments AB-AL, wherein the subject is no more than mildly depressed.

[Embodiment AO] The method of any of Embodiments AA or AD-AL, wherein the subject has a HAM-A total score≥20.

[Embodiment AP] The method of Embodiment AA or AB or any of Embodiments AD-AL, wherein the subject has a HAM-A anxious mood score≥2.

[Embodiment AQ] The method of Embodiment AA or AB or any of Embodiments AD-AL, wherein the subject has a HAM-A tension score of ≥2.

[Embodiment AR] The method of Embodiment AA or AB or any of Embodiments AD-AL, wherein the subject has a HAM-A total score≥20, a HAM-A anxious mood score≥2, and a HAM-A tension score of ≥2.

[Embodiment AS] The method of any one of Embodiments AA-AR, wherein the subject has a MADRS total score≤22.

[Embodiment AT] The method of any one of Embodiments AA-AS, wherein the subject has a MADRS inner tension score≥2, ≥3, ≥4, ≥5, or =6.

[Embodiment AU] The method of any one of Embodiments AA-AT, wherein the subject has a CGI-S score≥4.

[Embodiment AV] The method of any one of Embodiments AA-AU, wherein the anxiety disorder comprises one or a combination of feelings or symptoms selected from anxious mood, tension, fears, insomnia, intellectual, depressed mood, somatic (muscular), somatic (sensory), cardiovascular symptoms, respiratory symptoms, gastrointestinal symptoms, genitourinary symptoms, autonomic symptoms, and anxious behavior.

[Embodiment AW] The method of any one of Embodiments AA-AV, wherein the anxiety disorder comprises one or more anxious mood feelings or symptoms selected from worries, anticipation of the worst, fearful anticipation, and irritability.

[Embodiment AX] The method of any one of Embodiments AA-AW, wherein the subject has a HAM-A total score≥20, ≥22.5, ≥25, ≥27.5, ≥30, ≥32.5, or ≥35, and a HAM-A anxious mood score of ≥2, ≥3, or =4.

[Embodiment AY] The method of any one of Embodiments AA-AX, wherein the anxiety disorder comprises one or more tension feelings or symptoms selected from feelings of tension, fatigability, startle response, moved to tears easily, trembling, feelings of restlessness, and inability to relax.

[Embodiment AZ] The method of any one of Embodiments AA-AY, wherein the subject has a HAM-A total score≥20, ≥22.5, ≥25, ≥27.5, ≥30, ≥32.5, or ≥35, and a HAM-A tension score of ≥2, ≥3, or =4.

[Embodiment BA] The method of any one of Embodiments AA-AZ, wherein the anxiety disorder comprises one or more fears selected from fear of dark, fear of strangers, fear of being left alone, fear of animals, fear of traffic, and fear of crowds.

[Embodiment BB] The method of any one of Embodiments AA-BA, wherein the subject has a HAM-A total score≥20, ≥22.5, ≥25, ≥27.5, ≥30, ≥32.5, or ≥35, and a HAM-A fears score of ≥2, ≥3, or =4.

[Embodiment BC] The method of any one of Embodiments AA-BB, wherein the anxiety disorder comprises one or more insomnia feelings or symptoms selected from difficulty in falling asleep, broken sleep, unsatisfying sleep and fatigue on waking, dreams, nightmares, and night terrors.

[Embodiment BD] The method of any one of Embodiments AA-BC, wherein the subject has a HAM-A total score≥20, ≥22.5, ≥25, ≥27.5, ≥30, ≥32.5, or ≥35, and a HAM-A insomnia score of ≥2, ≥3, or =4.

[Embodiment BE] The method of any one of Embodiments AA-BD, wherein the anxiety disorder comprises one or more intellectual feelings or symptoms selected from difficulty in concentration and poor memory.

[Embodiment BF] The method of any one of Embodiments AA-BE, wherein the subject has a HAM-A total score≥20, ≥22.5, ≥25, ≥27.5, ≥30, ≥32.5, or ≥35, and a HAM-A intellectual score of ≥2, ≥3, or =4.

[Embodiment BG] The method of any one of Embodiments AA-BF, wherein the anxiety disorder comprises one or more depressed mood feelings or symptoms selected from loss of interest, lack of pleasure in hobbies, depression, early waking, and diurnal swing.

[Embodiment BH] The method of any one of Embodiments AA-BG, wherein the subject has a HAM-A total score≥20, ≥22.5, ≥25, ≥27.5, ≥30, ≥32.5, or ≥35, and a HAM-A depressed mood score of ≥2, ≥3, or =4.

[Embodiment BI] The method of any one of Embodiments AA-BH, wherein the anxiety disorder comprises one or more somatic (muscular) feelings or symptoms selected from pains and aches, twitching, stiffness, myoclonic jerks, grinding of teeth, unsteady voice, and increased muscular tone.

[Embodiment BJ] The method of any one of Embodiments AA-BI, wherein the subject has a HAM-A total score≥20, ≥22.5, ≥25, ≥27.5, ≥30, ≥32.5, or ≥35, and a HAM-A somatic (muscular) score of ≥2, ≥3, or =4.

[Embodiment BK] The method of any one of Embodiments AA-BJ, wherein the anxiety disorder comprises one or more somatic (sensory) feelings or symptoms selected from tinnitus, blurring of vision, hot and cold flushes, feelings of weakness, and pricking sensation.

[Embodiment BL] The method of any one of Embodiments AA-BK, wherein the subject has a HAM-A total score≥20, ≥22.5, ≥25, ≥27.5, ≥30, ≥32.5, or ≥35, and a HAM-A somatic (sensory) score of ≥2, ≥3, or =4.

[Embodiment BM] The method of any one of Embodiments AA-BL, wherein the anxiety disorder comprises one or more cardiovascular symptoms selected from tachycardia, palpitations, pain in chest, throbbing of vessels, fainting feelings, and missing beat.

[Embodiment BN] The method of any one of Embodiments AA-BM, wherein the subject has a HAM-A total score≥20, ≥22.5, ≥25, ≥27.5, ≥30, ≥32.5, or ≥35, and a HAM-A cardiovascular score of ≥2, ≥3, or =4.

[Embodiment BO] The method of any one of Embodiments AA-BN, wherein the anxiety disorder comprises one or more respiratory symptoms selected from pressure or constriction in chest, choking feelings, sighing, and dyspnea.

[Embodiment BP] The method of any one of Embodiments AA-BO, wherein the subject has a HAM-A total score≥20, ≥22.5, ≥25, ≥27.5, ≥30, ≥32.5, or ≥35, and a HAM-A respiratory score of ≥2, ≥3, or =4.

[Embodiment BQ] The method of any one of Embodiments AA-BP, wherein the anxiety disorder comprises one or more gastrointestinal symptoms selected from difficulty in swallowing, wind abdominal pain, burning sensations, abdominal fullness, nausea, vomiting, borborygmi, looseness of bowels, loss of weight, and constipation.

[Embodiment BR] The method of any one of Embodiments AA-BQ, wherein the subject has a HAM-A total score≥20, ≥22.5, ≥25, ≥27.5, ≥30, ≥32.5, or ≥35, and a HAM-A gastrointestinal score of ≥2, ≥3, or =4.

[Embodiment BS] The method of any one of Embodiments AA-BR, wherein the anxiety disorder comprises one or more genitourinary symptoms selected from frequency of micturition, urgency of micturition, amenorrhea, menorrhagia, development of frigidity, premature ejaculation, loss of libido, and impotence.

[Embodiment BT] The method of any one of Embodiments AA-BS, wherein the subject has a HAM-A total score≥20, ≥22.5, ≥25, ≥27.5, ≥30, ≥32.5, or ≥35, and a HAM-A genitourinary score of ≥2, ≥3, or =4.

[Embodiment BU] The method of any one of Embodiments AA-BT, wherein the anxiety disorder comprises one or more autonomic symptoms selected from dry mouth, flushing, pallor, tendency to sweat, giddiness, tension headache, and raising of hair.

[Embodiment BV] The method of any one of Embodiments AA-BU, wherein the subject has a HAM-A total score≥20, ≥22.5, ≥25, ≥27.5, ≥30, ≥32.5, or ≥35, and a HAM-A autonomic score of ≥2, ≥3, or =4.

[Embodiment BW] The method of any one of Embodiments AA-BV, wherein the anxiety disorder comprises one or more anxious behaviors selected from fidgeting, restlessness or pacing, tremor of hands, furrowed brow, strained face, sighing or rapid respiration, facial pallor, or swallowing.

[Embodiment BX] The method of any one of Embodiments AA-BW, wherein the subject has a HAM-A total score≥20, ≥22.5, ≥25, ≥27.5, ≥30, ≥32.5, or ≥35, and a HAM-A behavior at interview score of ≥2, ≥3, or =4.

[Embodiment BY] The method of any one of Embodiments AA-BX, wherein the subject has a HAM-A total score≥20, ≥22.5, ≥25, ≥27.5, ≥30, ≥32.5, or ≥35.

[Embodiment BZ] The method of any one of Embodiments AA-BY, wherein the subject has a HAM-A anxious mood score≥2, ≥3, or =4.

[Embodiment CA] The method of any one of Embodiments AA-BZ, wherein the subject has a HAM-A tension score≥2, ≥3, or =4.

[Embodiment CB] The method of any one of Embodiments AA-CA, wherein the subject has a MADRS total score≤22, ≤20, ≤18, ≤16, ≤14, ≤12, or ≤10.

[Embodiment CC] The method of any one of Embodiments AA-CB, wherein the subject is treatment naïve.

[Embodiment CD] The method of any of Embodiments AA-CB, wherein the subject has failed to adequately respond to prior buspirone or antidepressant therapy.

[Embodiment CE] The method of any of Embodiments AA-CB, wherein the subject has failed to adequately tolerate prior buspirone or antidepressant therapy.

[Embodiment CF] The method of Embodiment CD or CE, wherein the antidepressant therapy is selected from SSRIs, SNRIs, cyclic antidepressants, atypical antidepressants, and MAOIs.

[Embodiment CG] The method of any one of Embodiments AA-CF, wherein the treating comprises remitting the anxiety disorder.

[Embodiment CH] The method of any of Embodiments AA-CF, wherein the treating comprises a complete response to the anxiety disorder.

[Embodiment CI] The method of any one of Embodiments AA-CH, wherein the anxiety disorder is selected from generalized anxiety disorder (GAD), social anxiety disorder (SAD), obsessive-compulsive disorder (OCD), post-traumatic stress disorder (PTSD) and panic disorder.

[Embodiment CJ] The method of any one of Embodiments AA-CI, wherein the anxiety disorder is generalized anxiety disorder (GAD).

[Embodiment CK] The method of any one of Embodiments AA-CJ, wherein the therapeutically effective amount is 10-150 mg/day, 25-150 mg/day, 25-100 mg/day, 50-125 mg/day, 50-100 mg/day, or 50-75 mg/day, administered orally.

[Embodiment CL] The method of any one of Embodiments AA-CK, wherein the therapeutically effective amount is 10 mg/day, 15 mg/day, 20 mg/day, 25 mg/day, 50 mg/day, 75 mg/day, 100 mg/day, 125 mg/day, or 150 mg/day, administered orally.

[Embodiment CM] The method of any one of Embodiments AA-CL, wherein the therapeutically effective amount is administered once daily in the fed or fasted state.

[Embodiment CN] The method of any one of Embodiments AA-CM, wherein the ulotaront is administered as the hydrochloride salt.

### EXAMPLES

In the following examples, efforts have been made to ensure accuracy with respect to numbers (e.g., amounts, temperature, etc.) but some errors and deviations should be accounted for. The following examples are put forth so as to provide those of ordinary skill in the art with a complete disclosure and description of how the methods claimed herein are made and evaluated, and are intended to be purely exemplary of the disclosure and are not intended to limit the scope of what the inventors regard as their disclosure.

### Example 1. Effects of SEP-363856 on Mouse Marble Burying and Locomotor Activity in Mice

SEP-363856 was evaluated in the mouse marble burying test, which is sensitive to several anxiolytic and antidepressant drugs (Nicolas 2006). Male Swiss mice (5 weeks old, n=10/group) received a single administration of vehicle (po), SEP 363856 (0.3, 1, 3, or 10 mg/kg, po) or clobazam (8 mg/kg, i.p.) included as a positive control. Thirty minutes later mice were individually placed in transparent plastic cages with 5 cm of sawdust on the floor and 25 marbles grouped in the center of the cage. The number of marbles covered by sawdust (2/3^{rd} or more covered) was counted at the end of the 30 min test. Two days later, general locomotor activity was assessed in the activity meter test in the same animals. Mice were placed in an automated activity meter apparatus 30 minutes after dosing and the number of crossings recorded in 5-minute time bins for 30 minutes. Chlorpromazine (4 mg/kg, i.p.) was included as a positive assay control. SEP-363856 results in the marble burying test were analyzed by Kruskal-Wallis test followed by Mann-Whitney U test to determine group effects. Locomotor activity was analyzed by one-way ANOVA followed by Dunnett's post-hoc analysis. Effects of clobazam in the marble burying and activity meter tests were examined with the Mann-Whitney U test and unpaired student's t-test, respectively.

SEP-363856 significantly reduced the number of marbles buried at dose levels of 0.3, 3 and 10 mg/kg, po compared to vehicle-treated controls (Table 1). In the activity meter test, a modest, but significant decrease in the number of crossings was only observed at the highest dose (10 mg/kg, po) (Table 2). The results indicate that anxiolytic-like effects of SEP-363856 in the marble burying test are unlikely to be due to locomotor confounds at dose levels of ≤3 mg/kg.

TABLE 1
SEP-363856 Effects in the Mouse Marble Burying Test

| | Number of marbles buried |
|---|---|
| Group | (Mean ± SEM) |
| Vehicle | 23.9 ± 0.4 |
| Ulotaront 0.3 mg/kg, po | 18.8 ± 1.5^{a} |
| Ulotaront 1 mg/kg, po | 15.8 ± 3.0^{b} |
| Ulotaront 3 mg/kg, po | 13.0 ± 3.1^{a} |
| Ulotaront 10 mg/kg, po | 1.4 ± 0.9^{a} |
| Clobazam 8 mg/kg, ip | 0.5 ± 0.3^{a} |

^{a}p < 0.05,
^{b}p = 0.06 compared to vehicle

TABLE 2
SEP-363856 Effects in the Mouse Activity Meter Test

| | Number of crossings |
|---|---|
| Group | (Mean ± SEM) |
| Vehicle | 67.7 ± 3.9 |
| Ulotaront 0.3 mg/kg, po | 66.7 ± 6.1 |
| Ulotaront 1 mg/kg, po | 71.9 ± 4.2 |
| Ulotaront 3 mg/kg, po | 60.8 ± 7.4 |
| Ulotaront 10 mg/kg, po | 40.8 ± 7.9^{a} |
| Chlorpromazine 4 mg/kg, ip | 3.6 ± 0.7^{a} |

^{a}p < 0.05 compared to vehicle

### Example 2. a Phase 2/3, Randomized, Double-Blind, Parallel-Group, Placebo-Controlled, Flexible-Dose, Multicenter Study to Evaluate the Efficacy and Safety of SEP-363856 in the Treatment of Adults with Generalized Anxiety Disorder

This is a multicenter, randomized, double-blind, parallel-group, flexible dose, outpatient study evaluating the efficacy and safety of SEP-363856 flexibly dosed (50-75 mg/day) versus placebo over an 8-week Treatment Period in subjects with GAD. This study is projected to randomize approximately 434 subjects to 2 treatment groups (SEP-363856 [50-75 mg/day] or placebo) in a 1:1 ratio. Study drug is taken at approximately the same time each evening at bedtime and may be taken with or without food.

The study consists of 3 periods: Screening/Washout (up to 21 days), Treatment (8 weeks), and a Follow-up Visit (7 days after last study drug dose for subjects who discontinue prior to the Week 8 visit [Visit 7] or who complete the study).

During the Screening/Washout Period (up to 21 days), subjects are evaluated at the Screening Visit (Visit 1) to determine their eligibility to enroll in the study. During the screening/washout period, subjects are tapered off all psychotropic medications (except allowable Concomitant Medication) in a manner that is consistent with labeling recommendations and conventional medical practices, and fully discontinued at least 3 days or 5 half-lives (whichever is longer) prior to randomization.

During the Treatment Period (8 weeks), at Baseline (Day 1), subjects who have successfully completed the washout of prior medication and have met the eligibility criteria, are randomly assigned via a Randomization and Trial Supply Management (RTSM) system in a 1:1 ratio to one of two treatment arms: SEP-363856 (50-75 mg/day) or placebo. Dosing of study drug begins the evening of the Baseline Visit. Treatment continues once-daily, in the evening at bedtime, for the remainder of the 8-week Treatment Period.

All subjects receive 25 mg/day or matching placebo for the first three days of the Treatment Period and begin receiving 50 mg/day or matching placebo on Day 4. All subjects begin receiving 75 mg/day or matching placebo on Day 8. The investigator may request a dose reduction at any time after Week 1 (Visit 3) for reasons of safety or tolerability as judged by the Investigator. Subjects unable to tolerate the study medication after a dose reduction (blinded dummy dose reduction or actual dose reduction) are discontinued from the study.

At the End of Treatment Period, subjects have an End of Treatment (EOT) visit at Week 8 (Visit 7). Subjects who discontinue early from the study or complete the study are required to complete the Follow-up Visit 7 days (+2 days) post last dose of study drug. During the 1-week Follow-up Period, subjects should not be initiated on new treatment unless required due to the emergence of adverse events or necessary for the safety of the subject in the judgment of the Investigator. Upon completion of the Follow-up period, treatment may be initiated based on the judgment of the Investigator or the subject's primary care physician.

Primary Efficacy Objective: To evaluate the efficacy of flexible doses of SEP-363856 (50-75 mg/day) compared with placebo in subjects with GAD as measured by the Hamilton Anxiety Rating Scale (HAM-A) total score.

Secondary Efficacy Objective: To evaluate the efficacy of flexible doses of SEP-363856 (50-75 mg/day) compared with placebo in subjects with GAD as measured by the Clinical Global Impression-Severity (CGI-S) score.

Other Efficacy Objectives: (i) To evaluate the efficacy of flexible doses of SEP-363856 (50-75 mg/day) compared with placebo in subjects with GAD as measured by Proportion of subjects meeting HAM-A response criteria (≥50% improvement from Baseline score), Proportion of subjects meeting HAM-A remission criteria (≤7 total score), and Montgomery-Åsberg Depression Rating Scale (MADRS); (ii) To evaluate the effects of flexible doses of SEP-363856 (50-75 mg/day) on health-related quality of life and productivity as measured by: 36-Item Short Form Questionnaire (SF-36), Sheehan Disability Scale (SDS), and Healthcare Resource Utilization (HCRU); (iii) To evaluate medication satisfaction as measured by the Medication Satisfaction Questionnaire (MSQ). Primary Endpoint: Change from Baseline in HAM-A total score at Endpoint (Week 8).

Secondary Efficacy Endpoint: Change from Baseline in CGI-S score at Endpoint (Week 8).

Other Efficacy Endpoints: (i) Change from Baseline in: MADRS total score at Weeks 4 and 8, SDS total score at Weeks 4 and 8, SF-36 physical component score, mental component score, and subscales at Week 8; (ii) Change from Screening in MSQ score at Week 8; (iii) HAM-A response rate, defined as proportion of subjects with a 50% or greater improvement from Baseline in HAM-A total score at each scheduled visit and Endpoint (Week 8); (iv) HAM-A remission rate, defined as proportion of subjects with HAM-A total score≤7 at each scheduled visit and Endpoint (Week 8); (v) HCRU at Baseline and Week 8.

Select Subject Inclusion Criteria: To qualify for participation, subjects must meet all the following select inclusion criteria: (i) Male or female subject between 18 to 65 years of age (inclusive) at the time of consent; (ii) Subject meets The Diagnostic and Statistical Manual of Mental Disorders, Fifth Edition (DSM-5) criteria for GAD, as established by clinical interview (using the DSM-5 as a reference and confirmed using the Structured Clinical Interview for DSM-5-Clinical Trial Version (SCID-5CT) (subjects may have a comorbid DSM-5 based diagnosis of panic disorder, social anxiety disorder, or specific phobias provided the symptoms are secondary to GAD and not the primary focus of treatment); (iii) Subject must have a HAM-A total score≥20 and both HAM-A item 1 (anxious mood) score and HAM-A item 2 (tension) score of ≥2 on the clinician-administered HAM-A at Screening and Baseline; (iv) Subject must have a MADRS total score≤22 at Screening and Baseline; (v) Subject must have a CGI-S score≥4 at Screening and Baseline.

### REFERENCES CITED

Allgulander C, Hirschfeld R M, Nutt D J. Long-term treatment strategies in anxiety disorders. Psychopharmacol Bull. 2002; 36 Suppl 2:79-92.
Altamura A C, Dell'osso B, D'Urso N, Russo M, Fumagalli S, Mundo E. Duration of untreated illness as a predictor of treatment response and clinical course in generalized anxiety disorder. CNS Spectr. 2008; 13 (5): 415-422.
Baldwin D S, Anderson I M, Nutt D J, Bandelow B, Bond A, Davidson J R T, et al. Evidence-based guidelines for the pharmacological treatment of anxiety disorders: recommendations from the British Association for Psychopharmacology. J Psychopharmacol. 2005; 19 (6): 567-596.
Balon R, Starcevic V. Role of Benzodiazepines in Anxiety Disorders. Adv Exp Med Biol. 2020; 1191:367-388.
Bandelow B, Zohar J, Hollander E, Kasper S, Möller H J. World Federation of Societies of Biological Psychiatry (WFSBP) guidelines for the pharmacological treatment of anxiety, obsessive-compulsive and post-traumatic stress disorders: first revision. World J Biol Psychiatry. 2008; 9 (4): 248-312.
DuPont R L, Rice D P, Miller L S, Shiraki S S, Rowland C R, Harwood H J. Economic costs of anxiety disorders. Anxiety. 1996; 2 (4): 167-172.
First M B, Williams J B W, Karg R S, Spitzer R L. Structured Clinical Interview for DSM-5-Clinical Trials Version (SCID-5-C T); Arlington, V A, American Psychiatric Association, 2015.
Food and Drug Administration (U.S.). Discussion Document for Patient-Focused Drug Development Public Workshop on Guidance 3: SELECT, DEVELOP OR MODIFY FIT-FOR-PURPOSE CLINICAL OUTCOME ASSESSMENTS (including Appendices) (Published at Patient-Focused Drug Development Guidance: Methods to Identify What is Important to Patients and Select, Develop or Modify Fit-for-Purpose Clinical Outcome Assessments, Meeting Oct. 15-16, 2018) (“FDA 2018”)
Food and Drug Administration (U.S.). Patient-Focused Drug Development: Methods to Identify What Is Important to Patients Guidance for Industry, Food and Drug Administration Staff, and Other Stakeholders (October 2019) (“FDA 2019”)
Food and Drug Administration (U.S.). Patient-Focused Drug Development: Collecting Comprehensive and Representative Input; Guidance for Industry, Food and Drug Administration Staff, and Other Stakeholders (June 2020) (“FDA 2020”).
Garakani A, Murrough J W, Freire R C, Thom R P, Larkin K, Buono F D, et al. Pharmacotherapy of Anxiety Disorders: Current and Emerging Treatment Options. Front Psychiatry. 2020; 11:595584.
Gomez A F, Barthel A L, Hofmann S G. Comparing the efficacy of benzodiazepines and serotonergic anti-depressants for adults with generalized anxiety disorder: a meta-analytic review. Expert Opin Pharmacother. 2018 June; 19 (8): 883-894.
Goodwin R D, Gorman J M. Psychopharmacologic treatment of generalized anxiety disorder and the risk of major depression. Am J Psychiatry. 2002; 159 (11): 1935-1937.
Greenberg P E, Sisitsky T, Kessler R C, Finkelstein S N, Berndt E R, Davidson J R, et al. The economic burden of anxiety disorders in the 1990s. J Clin Psychiatry. 1999; 60 (7): 427-435.
Kessler R C, Petukhova M, Sampson N A, Zaslavsky A M, Wittchen H U. Twelve-month and lifetime prevalence and lifetime morbid risk of anxiety and mood disorders in the United States. International journal of methods in psychiatric research. 2012 September; 21 (3): 169-84.
Hamilton M. The assessment of anxiety states by rating. Br J Med Psychol 1959; 32:50-55.
Kong W, Deng H, Wan J, Zhou Y, Zhou Y, Song B, et al. Comparative Remission Rates and Tolerability of Drugs for Generalised Anxiety Disorder: A Systematic Review and Network Meta-analysis of Double-Blind Randomized Controlled Trials. Front Pharmacol. 2020; 11:580858.
Montgomery S A, Asberg M. A new depression scale designed to be sensitive to change. Br J Psychiatry. 1979; 134:382-389.
Nicolas L B, Kolb Y, Prinssen EPM. A combined marble burying-locomotor activity test in mice: a practical screening test with sensitivity to different classes of anxiolytics and antidepressants. Eur J Pharmacol. 2006; 547:106-115.
PCT Patent Publication No. WO 2020/118032
PCT Patent Publication No. WO2011/069063
PCT Patent Publication No. WO2019/161238
Pollack M H. Refractory generalized anxiety disorder. J Clin Psychiatry. 2009; 70 Suppl 2:32-38.
Ruscio A M, Hallion L S, Lim C C W, et al. Cross-sectional Comparison of the Epidemiology of DSM-5 Generalized Anxiety Disorder Across the Globe. JAMA Psychiatry. 2017; 74 (5): 465-475. doi: 10.1001/jamapsychiatry.2017.0056
Vernon M K, Revicki D A, Awad A G, Dirani R, et al. Psychometric evaluation of the Medication Satisfaction Questionnaire (MSQ) to assess satisfaction with antipsychotic medication among schizophrenia patients. Schizophr Res. 2010; 118:271-278.
Wittchen H U. Generalized anxiety disorder: prevalence, burden, and cost to society. Depress Anxiety. 2002; 16 (4): 162-171.
Yonkers K A, Dyck I R, Warshaw M G, Keller M B. Factors predicting the clinical course of generalised anxiety disorder. Br J Psychiatry. 2000; 176:544-549.
Yonkers K A, Warshaw M G, Massion A O, Keller M B. Phenomenology and Course of Generalized Anxiety Disorder. Br J Psychiatry. 1996; 168:308-313.

Throughout this application, various publications are referenced. The disclosures of these publications in their entireties are hereby incorporated by reference into this application in order to more fully describe the state of the art to which this disclosure pertains. It will be apparent to those skilled in the art that various modifications and variations can be made in the present disclosure without departing from the scope or spirit of the disclosure. Other embodiments of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

1. A method of treating an anxiety disorder in a non-schizophrenic human subject in need thereof who is no more than mildly depressed comprising administering to the subject a therapeutically effective amount of ulotaront or a pharmaceutically acceptable salt thereof, wherein the treatment results in a reduction in the subject's HAM-A total score compared to placebo.

2. A method of treating an anxiety disorder in a human subject in need thereof having a HAM-A total score≥20 comprising administering to the subject a therapeutically effective amount of ulotaront or a pharmaceutically acceptable salt thereof, wherein the treatment results in a reduction in the subject's HAM-A total score compared to placebo.

3. A method of treating an anxiety disorder in a human subject in need thereof having a HAM-A total score≥20, a HAM-A anxious mood score≥2, and a HAM-A tension score of ≥2, comprising administering to the subject a therapeutically effective amount of ulotaront or a pharmaceutically acceptable salt thereof, wherein the treatment results in a reduction in the subject's HAM-A total score compared to placebo.

4. The method of claim 2, wherein the treatment results in a clinically significant reduction in the subject's HAM-A total score compared to placebo, or wherein the treatment also results in a clinically significant reduction in the subject's CGI-S score compared to placebo.

5. (canceled)

6. The method of claim 2, wherein the treatment results in clinically significant reductions in the subject's HAM-A total score and CGI-S score compared to placebo eight weeks after commencing the administration, or wherein the treatment results in clinically significant reductions in the subject's HAM-A total score and CGI-S score compared to placebo four and eight weeks after commencing the administration.

7. (canceled)

8. The method of claim 2, wherein the treatment reduces the subject's HAM-A total score to ≤7, or wherein the treatment reduces the subject's HAM-A total score by ≥50%.

9. (canceled)

10. The method of claim 2, wherein the treatment reduces the subject's CGI-S score by ≥1 or ≥2, or wherein the treatment reduces the subject's CGI-S score to ≤3, or wherein the treatment reduces the subject's CGI-S score to ≤2, or wherein the subject has a CGI-S score≥4.

11. (canceled)

12. (canceled)

13. The method of claim 2, wherein the subject is non-schizophrenic.

14. The method of claim 2, wherein the subject is no more than mildly depressed.

15. The method of claim 1, wherein the subject has a HAM-A total score≥20.

16. The method of claim 2, wherein the subject has a HAM-A anxious mood score≥2; or wherein the subject has a HAM-A tension score of ≥2; or wherein the subject has a HAM-A total score≥20, a HAM-A anxious mood score≥2, and a HAM-A tension score of ≥2.

17. (canceled)

18. (canceled)

19. The method of claim 2, wherein the subject has a MADRS total score≤22; or wherein the subject has a MADRS inner tension score≥2, ≥3, ≥4, ≥5, or =6; or wherein the subject has a MADRS total score≤22, ≤20, ≤18, ≤16, ≤14, ≤12, or ≤10.

20. (canceled)

21. (canceled)

22. The method of claim 2, wherein the anxiety disorder comprises one or a combination of feelings or symptoms selected from anxious mood, tension, fears, insomnia, intellectual, depressed mood, somatic (muscular), somatic (sensory), cardiovascular symptoms, respiratory symptoms, gastrointestinal symptoms, genitourinary symptoms, autonomic symptoms, and anxious behavior.

23. The method of claim 2, wherein the anxiety disorder comprises:
one or more anxious mood feelings or symptoms selected from worries, anticipation of the worst, fearful anticipation, and irritability:
one or more anxious mood feelings or symptoms selected from worries, anticipation of the worst, fearful anticipation, and irritability; or
one or more tension feelings or symptoms selected from feelings of tension, fatigability, startle response, moved to tears easily, trembling, feelings of restlessness, and inability to relax; or
one or more fears selected from fear of dark, fear of strangers, fear of being left alone, fear of animals, fear of traffic, and fear of crowds; or
one or more insomnia feelings or symptoms selected from difficulty in falling asleep, broken sleep, unsatisfying sleep and fatigue on waking, dreams, nightmares, and night terrors; or
one or more intellectual feelings or symptoms selected from difficulty in concentration and poor memory; or
one or more depressed mood feelings or symptoms selected from loss of interest, lack of pleasure in hobbies, depression, early waking, and diurnal swing; or
one or more somatic (muscular) feelings or symptoms selected from pains and aches, twitching, stiffness, myoclonic jerks, grinding of teeth, unsteady voice, and increased muscular tone; or
one or more somatic (sensory) feelings or symptoms selected from tinnitus, blurring of vision, hot and cold flushes, feelings of weakness, and pricking sensation; or
one or more cardiovascular symptoms selected from tachycardia, palpitations, pain in chest, throbbing of vessels, fainting feelings, and missing beat; or
one or more respiratory symptoms selected from pressure or constriction in chest, choking feelings, sighing, and dyspnea; or
one or more gastrointestinal symptoms selected from difficulty in swallowing, wind abdominal pain, burning sensations, abdominal fullness, nausea, vomiting, borborygmi, looseness of bowels, loss of weight, and constipation; or
one or more genitourinary symptoms selected from frequency of micturition, urgency of micturition, amenorrhea, menorrhagia, development of frigidity, premature ejaculation, loss of libido, and impotence; or
one or more autonomic symptoms selected from dry mouth, flushing, pallor, tendency to sweat, giddiness, tension headache, and raising of hair; or
one or more anxious behaviors selected from fidgeting, restlessness or pacing, tremor of hands, furrowed brow, strained face, sighing or rapid respiration, facial pallor, or swallowing.

24. The method of claim 2, wherein the subject has a HAM-A total score≥20, ≥22.5, ≥25, ≥27.5, ≥30, ≥32.5, or ≥35, and:
a HAM-A anxious mood score of ≥2, ≥3, or =4; or
a HAM-A tension score of ≥2, ≥3, or =4; or
a HAM-A fears score of ≥2, ≥3, or =4; or
a HAM-A insomnia score of ≥2, ≥3, or =4; or
a HAM-A intellectual score of ≥2, ≥3, or =4; or
a HAM-A depressed mood score of ≥2, ≥3, or =4; or
a HAM-A somatic (muscular) score of ≥2, ≥3, or =4; or
a HAM-A somatic (sensory) score of ≥2, ≥3, or =4; or
a HAM-A cardiovascular score of ≥2, ≥3, or =4; or
a HAM-A respiratory score of ≥2, ≥3, or =4; or
a HAM-A gastrointestinal score of ≥2, ≥3, or =4; or
a HAM-A genitourinary score of ≥2, ≥3, or =4; or
a HAM-A autonomic score of ≥2, ≥3, or =4; or
a HAM-A behavior at interview score of ≥2, ≥3, or =4.

25-50. (canceled)

51. The method of claim 2, wherein the subject has a HAM-A total score≥20, ≥22.5, ≥25, ≥27.5, ≥30, ≥32.5, or ≥35; or wherein the subject has a HAM-A anxious mood score≥2, ≥3, or =4; or wherein the subject has a HAM-A tension score≥2, ≥3, or =4.

52-54. (canceled)

55. The method of claim 2, wherein the subject is treatment naïve.

56. The method of claim 2, wherein the subject has failed to adequately respond to prior buspirone or antidepressant therapy, or wherein the subject has failed to adequately tolerate prior buspirone or antidepressant therapy.

57. (canceled)

58. The method of claim 56, wherein the antidepressant therapy is selected from SSRIs, SNRIs, cyclic antidepressants, atypical antidepressants, and MAOIs.

59. The method of claim 2, wherein the treating comprises remitting the anxiety disorder, or wherein the treating comprises a complete response to the anxiety disorder.

60. (canceled)

61. The method of claim 2, wherein the anxiety disorder is selected from generalized anxiety disorder (GAD), social anxiety disorder (SAD), obsessive-compulsive disorder (OCD), post-traumatic stress disorder (PTSD) and panic disorder; or wherein the anxiety disorder is generalized anxiety disorder (GAD).

62. (canceled)

63. The method of claim 2, wherein the therapeutically effective amount is 10-150 mg/day, 25-150 mg/day, 25-100 mg/day, 50-125 mg/day, 50-100 mg/day, or 50-75 mg/day, administered orally; or wherein the therapeutically effective amount is 10 mg/day, 15 mg/day, 20 mg/day, 25 mg/day, 50 mg/day, 75 mg/day, 100 mg/day, 125 mg/day, or 150 mg/day, administered orally.

64. (canceled)

65. The method of claim 2, wherein the therapeutically effective amount is administered once daily in the fed or fasted state.

66. The method of claim 2, wherein the ulotaront is administered as the hydrochloride salt.
